(19) United States
(12) Patent Application Publication
(10) Pub. No.: US 2004/0236188 A1
(43) Pub. Date: Nov. 25, 2004

(54) Method and apparatus for monitoring using a mathematical model

(75) Inventors: George M. Hutchinson, Brookfield, WI (US); Paul S. Schluter, Whitefish Bay, WI (US)

Correspondence Address: GE MEDICAL SYSTEM, C/O FOLEY & LARDNER, 777 EAST WISCONSIN AVENUE, MILWAUKEE, WI, 53202-5367, US

(73) Assignees: GE Medical Systems Information; Technologies, Inc.

(21) Appl. No.: 10/440,747

(22) Filed: May 19, 2003

(51) Int. Cl. A61B 5/00

(52) U.S. Cl. 600/300

(57) ABSTRACT

A method and an apparatus for use in monitoring are disclosed. The method and system involve the use of a mathematical model. The method and system are particularly useful in the field of patient monitoring when using a physiological mathematical model. A mathematical model can be used to identify an abnormal condition. The mathematical model can also be used to generate an alarm. Also, the mathematical model can be used to generate a suggested treatment for correcting an abnormal condition if an abnormal condition should arise, especially abnormal conditions requiring relatively immediate attention.

### FIELD OF THE INVENTION

The invention relates to monitoring. More specifically, it relates to methods used to process data obtained during monitoring of a subject.

### BACKGROUND OF THE INVENTION

Monitors are used to monitor all sorts of variables to look for the occurrence of certain noteworthy events. Unfortunately, many of these monitors indicate that an event has occurred when in fact no significant event has occurred (false positive). A monitor that can reduce false positive rates without increasing false negative rates would be desirable.

Many subjects have unique characteristics. A value that would indicate an abnormal event for one subject, may be a normal value for another subject. A monitor that could use limits based on the characteristics of the subject would be preferable.

Sometimes a change in a value is a significant event. Other times no change in a value is more significant than a change in the value. It would be preferable to have a monitor that could recognize an event based on the absence of a change when the absence of a change is significant. It would be desirable to have a monitor that could both identify an event based on absence of a change when absence is significant, and presence of a change when presence is significant.

Additionally, in many emergency situations, when abnormal conditions are present, doctors must make quick decisions. Often times, a doctor must look through a large amount of information to make an appropriate decision. A system that can simplify a doctor's ability to make a decision would be preferable.

Also, some situations that involve emergency situations may be rare or uncommon for a particular physician. A system that could aid a physician in one of these circumstances would be preferable.

Additionally, in many on-going monitoring situations, a patient's potential physiological response is the most important feature for planning an intervention. A mathematical model used to aid in choosing the appropriate response would be beneficial. Further, excess data would likely not be necessary, and would likely add extra expense, to making the choices. Further, excess, marginally relevant, data would only slow down a decision making process. One such set of data that may not be as important for monitoring applications would be a patient's particular anatomic features. Since many patient monitoring decisions require quick decisions, and do not afford for big, expensive procedures to obtain and process data which may only be marginally relevant, a mathematical model used to monitor a patient preferably can operate based largely on a physiological mathematical model. Specifically, a mathematical model used to aid in the choice of alternative treatments for a patient being monitored preferably would not require incorporation of the anatomic features of the patient in order to aid in the decision making process (anatomic features being features such as the location of certain organs, number of ribs, locations of wounds, and other similar information as opposed to physical characteristics which include age, weight, height, race, sex, etc.).

The teachings hereinbelow extend to those embodiments which fall within the scope of the appended claims, regardless of whether they accomplish one or more of the above-mentioned needs.

### SUMMARY OF THE INVENTION

One embodiment is directed to a method for generating an alarm. The method comprises acquiring data from a subject and generating a comparison based on the data and a mathematical model representing the subject.

Another embodiment provides a method for generating an alarm using a medical monitoring device. The method comprises acquiring data from a patient and comparing the data to a physiological mathematical model. The comparison can then be used to identify an abnormal condition and generate an alarm. Information relating to the identified abnormal condition may also be displayed.

Another embodiment provides a method for treating a patient. The method comprises inputting physiological data relating to a patient, and determining an appropriate response based on the physiological data without using an anatomical mathematical model.

Another embodiment is directed to a medical monitoring system. The system comprises a data acquisition device configured to acquire physiological data relating to a patient. The system also comprises a processor configured to generate a comparison based on physiological data acquired by the data acquisition device and a mathematical model that considers effects of a treatment on a patient. The processor may also be configured to send an alarm signal based on the comparison.

Other principle features and advantages of the invention will become apparent to those skilled in the art upon review of the following drawings, the detailed description, and the appended claims.

### BRIEF DESCRIPTION OF THE DRAWINGS

FIG. 1 is a diagram of an exemplary embodiment of a system for monitoring according to one aspect of the invention where a subject is identified, where a monitor is connected to a network, and where a billing record can be generated based on the use of the monitor;

FIG. 2 is an exemplary illustration of a flow diagram for monitoring a subject using a mathematical model according to one aspect of the invention;

FIG. 3 is an exemplary illustration of a flow diagram for monitoring a subject according to another aspect of the invention where different alarms can be generated and where data is gathered from a plurality of monitors;

FIG. 4 is another exemplary embodiment of a system for monitoring a subject according to another aspect of the invention;

FIG. 5 is another exemplary illustration of a flow diagram for monitoring a subject using a mathematical model according to another aspect of the invention;

FIG. 6 is an exemplary illustration of a comparison between an acquired data stream and a simulated data stream according to one aspect of the invention.

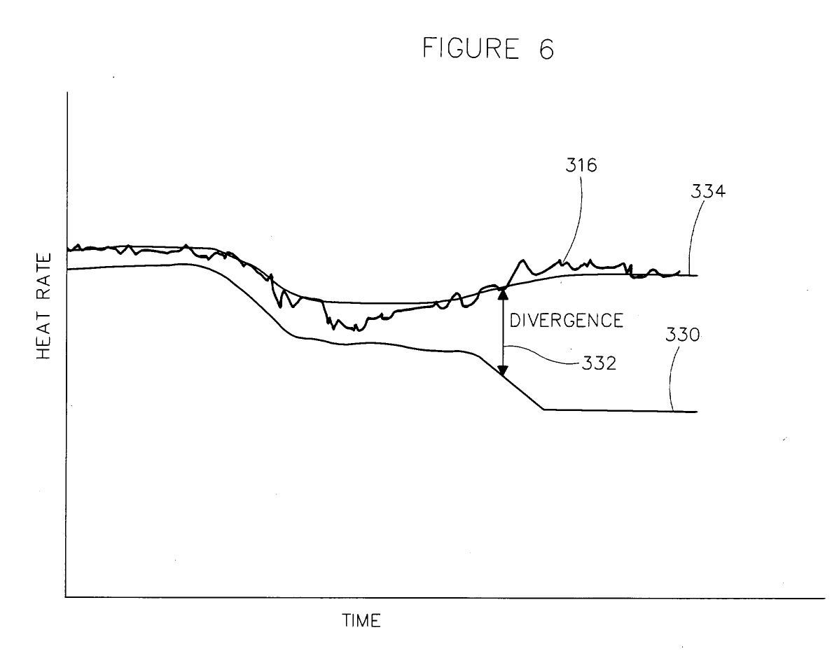

### DETAILED DESCRIPTION OF THE PREFERRED EMBODIMENTS

In the following description, for purposes of explanation, numerous specific details are set forth in order to provide a thorough understanding of the exemplary embodiments of the present invention. It will be evident, however, to one skilled in the art that the exemplary embodiments may be practiced without these specific details. In other instances, well-known structures and devices are shown in block diagram form in order to facilitate description of the exemplary embodiments.

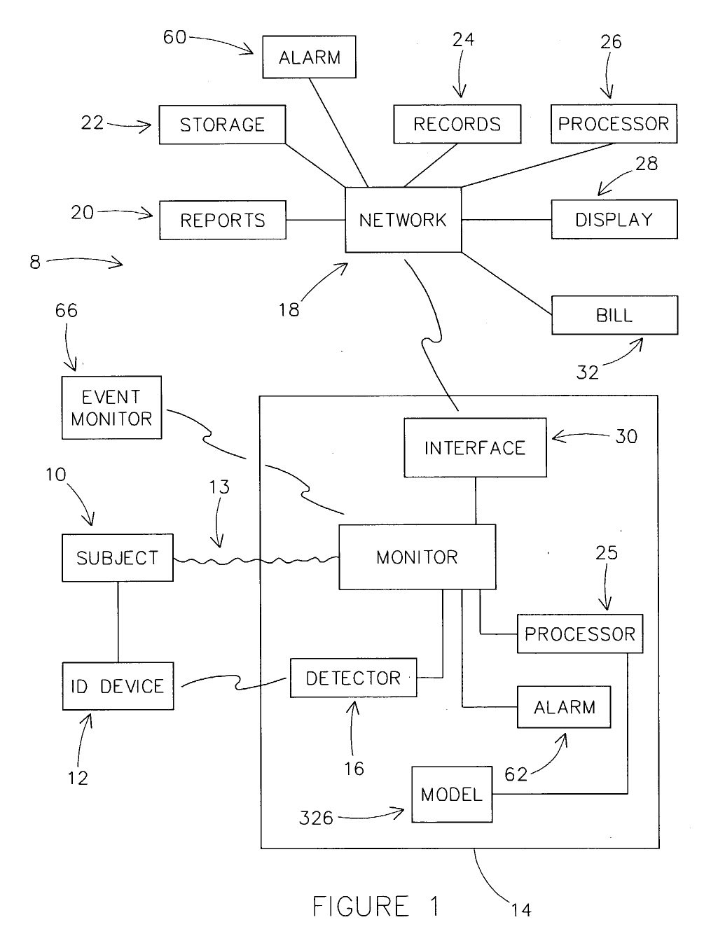

Referring first to FIG. 1, a monitoring system 8 comprises a monitor 14 and a network 18. Monitor 14 also comprises a network interface 30 that allows transfer of data to and from network 18. Network interface 30 is preferably configured to allow wireless transfer of data. More preferably, network interface 30 is configured to transmit data using a radio frequency. Network interface 30 may directly facilitate transfer of data across a network for the monitor, or may facilitate transfer of data by coupling the monitor to some other device that can directly facilitate transfer.

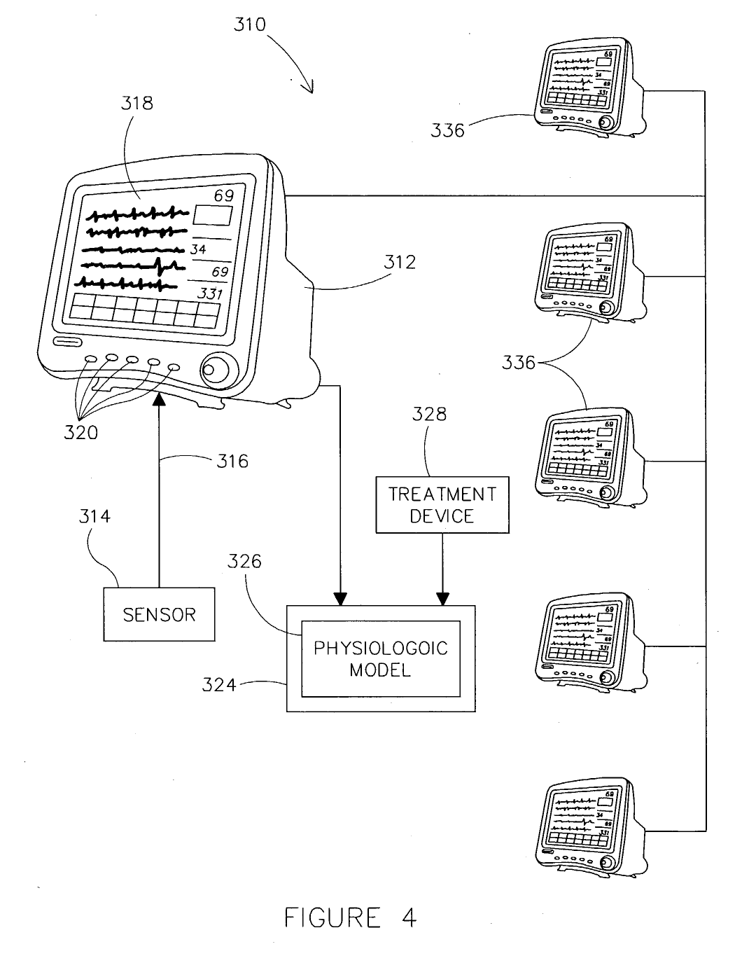

The data transferred from monitor 14 to network 18 can be raw data and/or processed data. Also, data can be transferred to monitor 14 to aid, configure, or operate a function of monitor 14, or can serve some other purpose relating to monitor 14. For instance, a mathematical model 326 (FIG. 4) relating to a specific subject can be transferred to monitor 14 using network 18.

Data acquisition device 13 acquires data from subject 10. The data acquired by data acquisition device 1 3 is preferably physiological data from a patient. Processor 25 can be configured to generate a comparison based on data acquired by the data acquisition device and a mathematical model 326, and send an alarm signal based on the comparison. For instance, processor 25 may be configured to send an alarm signal if the physiologic values from the patient deviate beyond a threshold amount or a significant amount from what would normally be expected based on the mathematical model.

Processor 25 may be any signal processing circuitry, such as one or more microprocessors in combination with program logic stored in memory. Processor 25 may be made of a series of sub-processors where each sub-processor performs one of the functions of processor 25. Further, processor 26 may perform the functions of processor 25. Further still, processor 26 and processor 25 may be sub-processors of another processor that is responsible for the various functions.

Physiological mathematical models 326 allow for simulation of a variety of human body compartments and their reaction to treatment processes. A mathematical model that considers effects of events on a subject is any model that represents the working of a subject mathematically, including taking into account what data would be expected from data acquisition device 13 given the events affecting subject 10. The model may be constructed using finite element alanlysis techniques. For example, a physiological mathematical model 326 may operate by dividing a patient's bodily system into compartments and tries to represent mathematically what happens, physiologically, in each compartment and how the various compartments interact and respond to medical treatments. This may be used to calculate predicted values for physiologic data if a given event occurs, such as laying in bed, eating, breathing, moving, being injected with anesthesia, taking medicine, reacting to a stimulus, reacting to a therapy, etc. The model 326 preferably can generate predicted values for a plurality of events.

The mathematical model 326 can be generic, but is preferably tailored to take into account various properties of the subject. For a patient, the model 326 may take into account age, weight, and/or other criteria. Additionally, the model may take into account properties of a subject by incorporating empirical data relating to the subject. Some possible empirical data to be incorporated could include the results of imaging scans, tests run on the subject, physiological inputs, and various other patient data.

There are a number of ways to tailor mathematical model 326 to a subject 10. A user can enter the subject's attributes into the mathematical model 326. Such information can be received directly from a subject's file. In addition to those attributes commonly found in a subject's file 10, the file may contain data from registering a pre-treatment data stream from subject 10 to incorporate into the attributes of the subject 10.

One example of a mathematical model that may be used as a base model of a patient monitoring system to monitor the physiology of a patient is BODY Simulation for Anesthesia. BODY Simulation is a multi-media interactive anesthesia trainer that has been implemented on a PC. It simulates a patient, an anesthesia workstation, a ventilator and gas delivery circuit, parts of the operating room, and even some operating room personnel. Body Simulation for Anesthesia is based on mathematical models of physiology and pharmacology. When affected by a stimulus (drugs, gases, pain, etc.), the patient's response is calculated to be as close as possible to that of an average person. This is done using a complex set of mathematical equations.

Body Simulation can be used to produce real time data plots allowing a user to see different clinical and physiologic parameters graphically displayed. Graphics of drug concentrations and drug mass in 16 different body compartments may be viewed. Dynamic gas displays and X-Y plots of respiration are available. These tools allow the user to see the pressures, flows, resistance, and compliance in the heart, blood vessels, lungs and other organs, as well as drug concentrations and/or masses in the compartments. Scientific data may be viewed in real time as events are occurring during the case.

The mathematical model 326 may also be adjustable. One manner in which the model 326 may be adjustable is based on results of monitoring. For instance, if the model 326 keeps generating false alarms, the model may adjust to better suit the subject, to be more tolerant, and/or in some other manner to reduce the likelihood of false alarms.

The model 326 may also be changeable. For instance, the model may be changeable in that if a new drug has been studied with respect to the model, an upgrade can be added to take into account the effects of the new drug. Also, the model may be changeable in that one portion of the model may be used in one instance, but other portions of the model may be used in other instances. This may allow the relevant portions to be applied, while not requiring the lengthy procedure of running through every portion of the model in every instance.

The alarm signal generated by processor 25 may be based on a tolerance factor where a larger difference is allowed if the tolerance factor is higher. The tolerance factor can be based on a number of different criteria. For example, the tolerance factor may be adjusted by a user, may be adjusted based on information relating to subject 10, and/or may be adjusted based on the amount of data inputted from subject 10 (the more data that has been inputted, the more likely the mathematical model accurately represents the subject). The tolerance factor may change over time and may be different for different applications of the model to subject 10.

Further, the alarm signal sent by processor 25 may be sent to an alarm signaling device 62 physically connected to processor 25, or may be sent to an alarm signaling device 60 located remote from processor 25. Remote alarm signaling device 60 may be a part of a pager or some other type of communication device. Remote alarm signaling device 60 could also be located at a discrete location such as at a nurse's station in a health care facility. The signal from processor 25 would then cause alarm signaling devices 60 and 62 to generate an alarm.

The alarm generated by alarm signaling devices 60 and 62 may take on any form including, but not limited to, an audible sound, a visual indicator, and/or a vibrating alert. The alarm generated by alarm signaling devices 60 and 62 can include a message indicating the reason for the alarm. The alarms generated by alarm signaling devices 60 and 62 could also be differentiated based on a number of criteria including the type and severity of the event causing the alarm. Further, if a system has more than one alarm signaling device, the device that signals the alarm could be differentiated based on a number of criteria including the type and severity of the event underlying the alarm.

Processor 25 can also be configured to generate information useful for formulating a response if an abnormal condition (one that might set off an alarm) is identified. An abnormal condition can be identified in a number of manners by a number of different techniques.

Possible reasons that an abnormal condition exists could include an actual abnormal condition, a malfunction in equipment, or improper set up of the equipment (originally or caused to be improper by some later event—such as patient movement).

Processor 25 can process the data inputted from various sensors and display information based on the inputted data when an abnormal condition exists. The information displayed could be listing the data that resulted in the determination that an abnormal condition exits, could be displaying the reasons that an abnormal condition was indicated (such as the data and the calculations made based on the data), could be suggesting reasons why an abnormal condition might exist, could be suggesting an appropriate reaction to the fact that an abnormal condition was indicated, and/or could be some other information relating to the abnormal condition.

In a health care setting, a mathematical model is preferably used to determine an appropriate response to the abnormal condition that was identified from the monitoring of the patient. Such an abnormal condition would likely have an immediate adverse effect on the patient. A mathematical model can be used to identify the response that will best alleviate the abnormal condition by determining the likely effect of administering different treatments in response to the abnormal condition. Such a system could include balancing the longer term effectiveness of a treatment against the short term need to alleviate the immediate adverse effects of the abnormal condition.

When applied to a patient, processor 25 can input various physiological data relating to a patient to look for an abnormal condition. The physiological data that is inputted can be applied to a physiological or pathophysiological based mathematical model. Such a model may be useful for ongoing monitoring of patients such as occur in a critical care facility.

Storage 22 may include a database that stores a mathematical model. The stored mathematical model may be a generic model, or may be a model that had previously been customized to subject 10. Data from storage 22 may be transferred to monitor 14, and data from monitor 14 may be transferred to storage 22.

Monitoring system 8 may also include an event monitor 66. Event monitor 66 can monitor the occurrence of an event that might affect the predicted values based on the mathematical model being used by processor 25. For example, a patient may receive medication intravenously so event monitor 66 can monitor the rate, and using the concentration of the medication, also monitor the amount of medication being administered. Also, event monitor 66 could be used to indicate that subject 10 is moving or lying down, and even the rate at which subject 10 is moving or for how long subject 10 has been laying down. There are also a large number of other events that could be monitored by event monitor 66. Event monitor 66 would then send a signal based on its monitoring of subject 10. The event monitor signal could then be included in the calculation of the predict values based on the mathematical model.

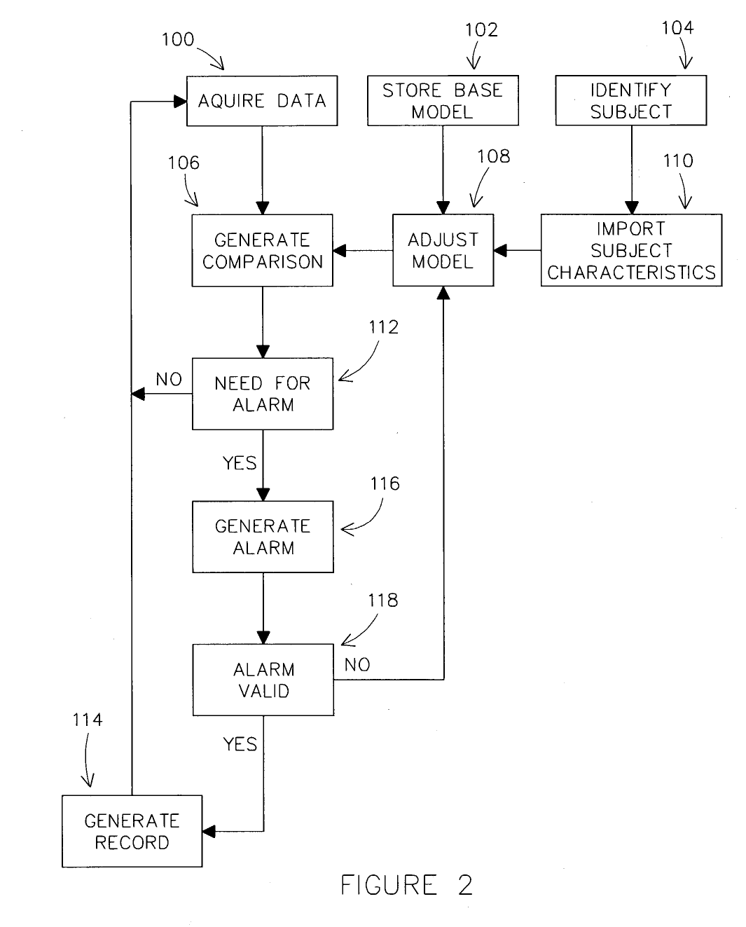

Referring to FIG. 2, a process for monitoring a subject using a mathematical model includes identifying a subject at step 104. The identification at step 1 04 can be performed manually (an operator enters a patient ID code into monitor 14, an operator inserts a patient ID card, etc.), or automatically (patient is identified wirelessly using a wireless detector). Based on the subject identified, characteristics of the identified subject can be imported at step 110. These characteristics can be used along with the stored base mathematical model to form an adjusted model at step 108. This can be done by modifying parameters of the mathematical model to reflect characteristics of the subject. The base mathematical model stored at step 102 can be a generic model or can be a model that had previously been tailored to the subject. For a patient, the base mathematical model preferably includes a physiological mathematical model.

Also, data is acquired from the subject at step 100. For a patient, the data preferably includes physiological data collected by a monitor. The data can be from one source or can be from multiple sources. A comparison is generated at step 106 based on the adjusted model from step 108 and the data acquired at step 100. The comparison preferably includes comparing at least one value of the acquired data to a value predicted using the mathematical model, and determining the difference between the values.

At block 112 the comparison generated at block 106 is used to determine whether an alarm should be generated. Determining whether an alarm should be generated can be based on any number of criteria. Further, different alarm types/levels can be generated based on different criteria. If an alarm is not generated then data is acquired at step 100. If an alarm should be generated, then an alarm is generated at step 116.

A determination is then made at step 118 as to whether the alarm is a valid alarm. This determination can be made by a user who sends an input if the alarm should not have been generated, can be made by determining if other sources for monitoring the subject indicate that an alarm should be generated, can be made using some combination of these criteria, or can be made using some other criteria. If the alarm is not valid, the mathematical model is adjusted at step 108 in an attempt to make the model function as a better predictor of appropriate alarms. If the alarm is valid, a record of the alarm is made at step 114 and data is acquired at step 100.

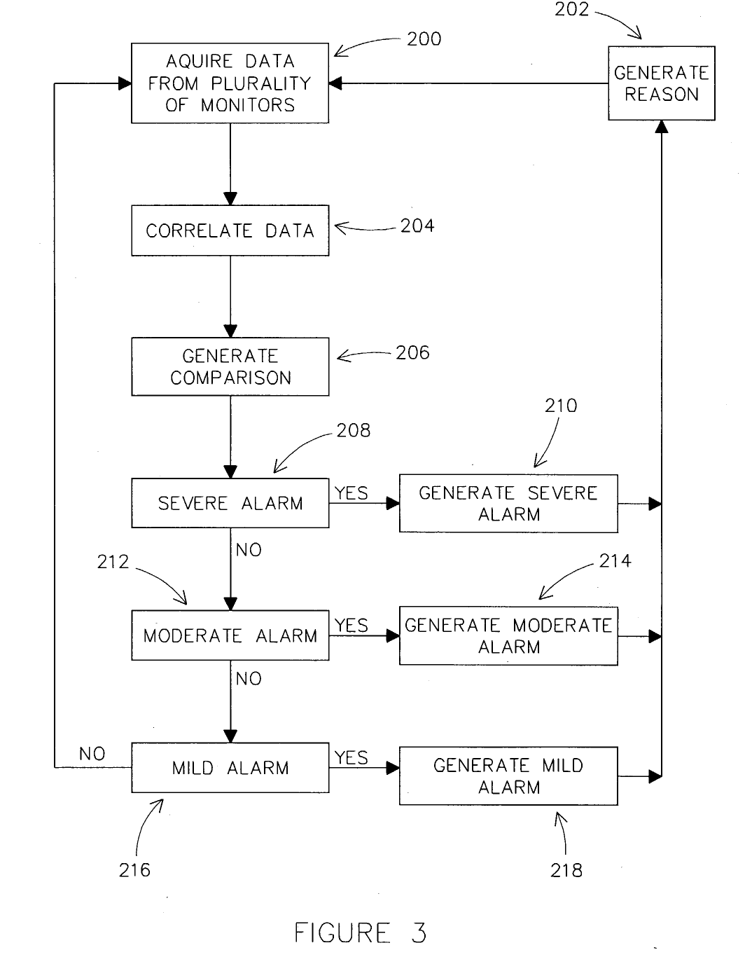

Referring to FIG. 3, data relating to a subject is acquired at block 200 from a plurality of monitors. The data from the plurality of monitors is correlated at block 204 to form a correlated data set. The correlated data set could refer to only one monitored characteristic of the subject, or could refer to multiple monitored characteristics of the subject. At block 206 the correlated data is used to generate a comparison between the correlated data set and a mathematical model of the subject. The comparison could comprise comparing the data from each of-the monitors individually to predicted values based on the mathematical model, or may comprise comparing the correlated data set as a whole to predicted values based on the mathematical model.

At block 208, the comparison of block 206 is used to determine if conditions are severe enough to generate a severe alarm at block 210. If conditions are not severe enough, the comparison of block 206 is used at block 212 to determine if conditions are such that a moderate alarm should be generated at block 210. If conditions do not warrant a moderate alarm, the comparison of block 206 is used at block 216 to determine if conditions are such that a moderate alarm should be generated at block 218. The severity of the alarm generated may depend on the amount of difference between the predicted value and the data, may be based on the number of data values that differ from the predicted values, etc.

If no alarm is generated, data is acquired at block 200. If an alarm is sent at blocks 210, 214, or 218, an indication of the reason for the alarm is generated at block 202. The indication could be made in any number of forms. Further, the indication may indicate what values are not appropriate and/or what monitors are giving readings indicating the alarm. Further still, the values leading to the alarm may be grouped together to give a user a better indication of the reason for the alarm (rather than needing to view a plurality of different locations to find the appropriate values).

Referring now to FIG. 4, patient physiologic monitoring assembly 310 includes a controller 312 in communication with a patient sensor 314 in order to receive a real-time physiologic data stream 316. It is contemplated that the patient sensor 314 and real-time physiologic data stream 316 may encompass a wide variety of patient monitoring physiologic characteristics. These characteristics include, but are not limited to, heart rate, arterial blood pressure, StO2, CO2, EtC2, respiratory rate, and a variety of other patient physiologic responses. It should be understood that a wide variety of such responses and sensors 314 designed to receive them could be used. Similarly, a host of amplifiers, filters, and digitization elements may be utilized in combination with the sensors 314 as would be understood by one skilled in the art. The controller 312 may be utilized in combination with a variety of interactive elements such as a display 318 and control features 320 as would be comprehended by one skilled in the art.

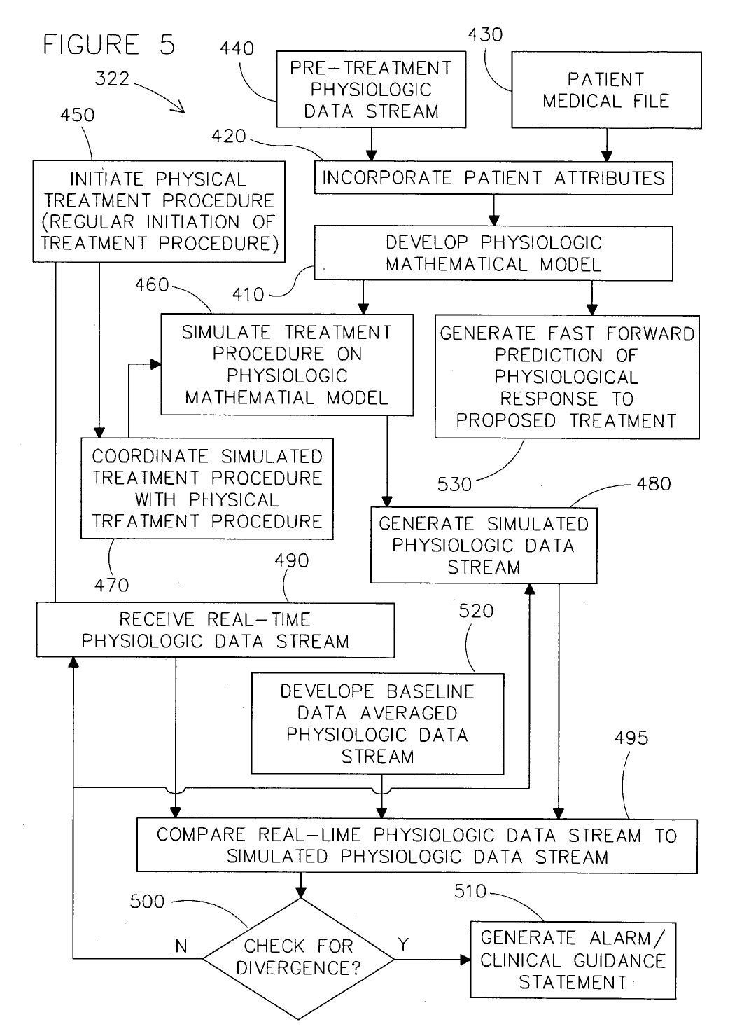

The controller 312 includes a logic 322 adapted to perform a plurality of functions as is illustrated in FIG. 5. It should be understood that although the terms controller 312 and logic 322 are utilized in the singular vernacular, a plurality of individualized controllers 312 and logics 322 could be used and are contemplated as incorporated into the chosen vernacular. By way of example, an independent physiologic emulation system 324 may be utilized to perform various functions. The logic 322 is adapted to develop a physiologic mathematical model 410 of the patient.

The logic 322 registers initiation of a treatment procedure 450. A wide variety of treatment procedures may be used. By way of example, one contemplated treatment procedure anticipates the administration of anesthesia to a patient prior to surgery. Other treatment procedures, however, may encompass a wide range of procedures including, but not limited to, drug injections, gas treatment, and even simply monitored care. The initiation of the treatment procedure 450 is intended to encompass a plurality of simultaneous individual treatments. The initiation of treatment procedure 450 is coordinated with a simulation of the treatment procedure on a physiologic mathematical model 460. As stated, the physiologic mathematical model 326 is a simulation of a human anatomical system that allows simulation of treatment and predictive responses to such treatment.

It is contemplated that a separate step in logic 322 of coordinating the simulated treatment with the physical treatment procedure 470 may also be incorporated. The coordination logic 470 is intended to encompass a wide variety of embodiments.

In one embodiment, it is contemplated that a clinician after selecting a treatment and parameters within the mathematical model 326 will activate the simulation procedure at approximately the same time as the physical procedure is beginning.

In another embodiment, however, it is contemplated that the simulated procedure (mathematical model 326) can be placed in communication with a treatment device 328, or group of such devices, such that the activation of the treatment device 328 can automatically effectuate the start of simulated treatment. In still another contemplated embodiment, the communication between the treatment device 328 and the mathematical model 326 allows for accurate real-time treatment information to be supplied to the mathematical model 326. For example, type, quantity, and flow rate of anesthesia may be automatically communicated from the treatment device 328 to the mathematical model 326 such that the simulated treatment accurately reflect the physical treatment without requiring excessive interaction from a clinician.

The mathematical model 326 is utilized to generate a simulated physiologic data stream 480 in response to the simulated treatment 460. It should be understood that the simulated physiologic data stream 330 need not represent a moment-to-moment exact prediction of patient physiologic data but may also be represented by ranges of predicted responses over time. The logic 322 also is adapted to receive a real-time physiologic data stream 490 from the patient. As stated, the real-time physiologic data stream 316 is intended to comprise a wide variety of different patient physiologic characteristics. The logic 322 is adapted to compare the real-time physiologic data stream with the simulated physiologic data stream 495. This allows the real-time physiologic data stream 316 to be compared to the simulated physiologic data stream 330 to verify the patient is responding to treatment as predicted by the mathematical model 326. The logic 322 then checks for divergence 500 between the real-time physiologic data stream 316 and the simulated physiologic data stream 330 to determine if the patient's response to treatment is different from that predicted by the mathematical model 326. If a divergence 332 is discovered, the logic 322 is adapted to generate an alarm warning 510. The alarm warning is intended to comprise both audible alarms as well as clinical guidance statements.

It is contemplated that a wide variety of approaches to checking for divergence 500 may be utilized. In one contemplated embodiment, the divergence 332 may simply represent a hard threshold value in relation to the simulated physiologic data stream 330 that once crossed by the real-time physiologic data stream 316 sets off the alarm warning. In other embodiments, the divergence 332 may be registered when the real-time physiologic data stream 316 begins to move in a direction opposite that predicted by the simulated physiologic data stream 330. Thus, if the simulated physiologic data stream 330 predicts heart rate to drop and the real-time physiologic data stream 316 rises or remains the same, a divergence 332 is registered by the logic 322. As a practical example, if a patient is undergoing surgery, anesthesia is commonly given. The patient's blood pressure commonly drops quickly in response to the anesthesia. During a portion of the surgery, however, the surgery is aggravating and effectuates a rise in blood pressure. Thus, the effective blood pressure would remain the same. Normal monitoring systems have no way to determine that this non-change in blood pressure should generate a warning alarm (as the drop in blood pressure due to anesthesia is desired). Here, when the blood pressure remains the same, a divergence 332 is registered and an alarm can be sounded 510.

A variety of features intended to reduce the occurrence of undesired alarm warnings may also be incorporated. One such feature contemplates the development of a baseline data-averaged physiologic data stream 520. The baseline data averaged value 334 is a mean rate of the real-time physiologic data stream 316. The term data averaged and mean rate are intended to encompass any of a wide variety of data averaging and tracking techniques. By way of example, one such embodiment compares each new physiologic data sample and compares it to the running baseline 334 and increments or decrements the next point in the baseline 334 by a predetermined amount. Thus, utilizing this technique, or a variety of others, the baseline data averaged value 334 can track true physiologic changes that are consistent over time.

Additionally, the use of a baseline data averaged value 334 is beneficial in ignoring noise and other generated artifacts. In embodiments utilizing the baseline data averaged values 334, the comparison of the real-time physiologic data stream to the simulated physiologic data stream 490 is accomplished by comparing the simulated physiologic data stream 330 to the baseline data averaged value 334. Although a single method of reducing unwanted alarms has been disclosed, a wide variety of methods and approaches may be utilized.

A variety of features could be added to extend the usefulness of the monitoring system 8 within a medical setting. One such additional feature is achieved by adapting the logic 322 to generate a prediction of simulated physiologic response to proposed treatment 530. This allows a clinician to check what a patient's response to a treatment will be prior to actual initiation of treatment 450. The unique advantage of this feature is that it allows a clinician to access such predictive capabilities directly from the monitoring system 310 in the treatment room during treatments such as operations. Thus, instantaneous predictive advice is available during surgery and other treatment options that has previously been unavailable.

An additional feature comprises a plurality of networked monitors 336 in communication with the monitoring system 310. These networked monitors 336 allows the patient to be moved to any of the monitors within the network and still retain the ability to compare the real-time physiologic data stream 316 with the simulated physiologic data stream 330. By way of example, a patient may be subjected to anesthesia during a surgical procedure. After the surgical procedure, the patient is commonly moved into a recovery room. Through the use of the networked monitors 336 in communication with the monitoring system 310, mathematical model 326 may continue to produce a simulated physiologic data stream 330 that can be compared with the real-time physiologic data stream 316. Therefore, as the model predicts the adjustment of the physiologic data in response to the gradual emergence from the effects of the anesthesia, it can be compared to the real-time physiologic data stream 316 to monitor if the patient experiences problems in recovery. Thus, if the patient does not properly emerge from the anesthesia as desired, a warning alarm can be sounded to draw a clinician for further analysis. Although a single example has been provided, it should be understood that a wide variety of procedures may make use of the networked monitors 336.

Referring again to FIG. 1, monitor 14 comprises an identity detector device 16 configured to identify a subject 10. Identity detector device 16 can identify subject 10 by detecting an identification device 12 associated with a subject of interest 10. Identification device 12 can be a card or other object associated with the subject. Identification device 12 is can be configured to allow wireless detection by identity detector device 16.

Network 18 can be any type of network across which data can be transferred. For example, network 18 can be a local area network, a wide area network, and an internet. Network 18 is coupled to a report generator 20, a data storage device 22, a record keeping device 24, a processor, and a display. Report generator 20 can generate a report based on, data storage device 22 can store, record keeping device 24 can make or add to a record based on, processor 26 can process, and display 28 can display data acquired by a data acquisition device 1 3 of monitor 14.

Additionally, a bill generator 32 can generate a bill based on the use of monitor 14. Bill generator 32 can generate a bill for the use of monitor 14, or can integrate the use of monitor 14 into a larger bill to be sent. Bill generator 32 can also monitor the usage of monitor 14, and generate reports based on usage of monitor 14. Bill generator 32 can also be used to send a notice to a person across network 18 indicating that monitor 14 is being used and billed. People that may desire receiving such a notice might include a patient's primary physician, a treating physician, an insurance carrier, and a patient. Delivering a notice to an insurance carrier may allow faster approval for sudden, unexpected usage of monitor 14. This would allow a hospital to collect funds sooner, and would allow a patient to worry less about obtaining coverage after treatment. Once the bill is generated, it can then be sent physically or electronically to a recipient. The recipient may be a computer at an insurance company that calculates the extent of coverage and the amount to be paid based on the usage of monitor 14.

The invention has been described with reference to various specific and illustrative embodiments and techniques. However, it should be understood that many variations and modifications may be made while remaining within the spirit and scope of the invention. For instance, while the invention is particularly useful for patient monitoring, some aspects of the invention are applicable to other monitoring activities.

What is claimed is:

1. A method for generating an alarm, comprising:
acquiring data from a subject; and
generating a comparison based on the data and a mathematical model representing the subject.

2. The method of claim 1, further comprising generating an alarm based on the comparison.

3. The method of claim 1, further comprising identifying an abnormal condition based on the comparison.

4. The method of claim 3, further comprising displaying information relating to an abnormal condition that has been identified.

5. The method of claim 4, wherein the information displayed is a reason that the abnormal condition was identified.

6. The method of claim 4, wherein the information displayed is a suggested response to the identification of the abnormal condition.

7. The method of claim 1, wherein the data is data relating to a physiological characteristic.

8. The method of claim 1, wherein the mathematical model comprises a physiological mathematical model.

9. The method of claim 1, wherein comparing the data to the mathematical model comprises determining a degree of difference between a predicted value predicted based on the model and a data value based on the acquired data.

10. The method of claim 9, wherein the alarm generated depends on the degree of difference between the predicted value and the data value.

11. The method of claim 2, further comprising generating an alarm based on the comparison and indicating a reason for the alarm.

12. The method of claim 1, further comprising modifying parameters of the mathematical model to reflect characteristics of the subject.

13. The method of claim 12 , further comprising obtaining characteristics of the subject from a database.

14. The method of claim 1, further comprising identifying a subject using an identification device.

15. A method for generating an alarm in a medical monitoring device, comprising:
acquiring data from a patient; and
comparing the data to a physiological mathematical model.

16. The method of claim 15, further comprising generating an alarm based on the comparison of the data to the mathematical model.

17. The method of claim 15, further comprising identifying an abnormal condition based on the comparison.

18. The method of claim 17, further comprising displaying information relating to an abnormal condition that has been identified.

19. The method of claim 15, wherein the mathematical model of the patient models effects of a treatment selected from a group consisting of an administered drug and an administered therapy.

20. The method of claim 15, further comprising identifying a subject using an identification device.

21. The method of claim 15, wherein comparing the data to the mathematical model comprises determining a degree of difference between a predicted value predicted based on the model and a data value based on the data.

22. The method of claim 23, wherein the alarm generated depends on the degree of difference between the predicted value and the data value.

23. The method of claim 15, further comprising generating a record of an alarm that is generated.

24. The method of claim 15, wherein acquiring data from a patient comprises acquiring physiological data from a plurality of monitors.

25. A medical monitoring system comprising:
a data acquisition device configured to acquire physiological data relating to a patient; and
a processor configured to generate a comparison based on the physiological data acquired by the data acquisition device and a predicted value predicted by a physiological mathematical model, and configured to send an alarm signal based on the comparison.

26. The system of claim 25, further comprising an alarm signaling device that is responsive to the alarm signal from the processor.

27. The system of claim 25, further comprising a network interface that facilitates transfer of data across a network.

28. The system of claim 27, wherein the network interface facilitates the transfer of data relating to the physiological mathematical model.

29. The system of claim 25, further comprising an identity detection device configured to identify the patient.

30. The system of claim 25, further comprising a bill generator configured to generate a billing record based on the use of the system.

31. The system of claim 25, wherein the processor determines a degree of difference between the predicted value and a data value that is based on the physiological data.

32. The system of claim 31, wherein the processor generates a different alarm signal based on the degree of difference between the predicted value and the data value.

33. The system of claim 25, wherein the processor is configured to send an indication signal indicating the reason that the alarm signal is generated when the alarm signal is generated.

34. The system of claim 25, wherein the processor is configured to establish the mathematical model based on characteristics of the patient that are stored in a database.

35. A method for providing guidance relating to treatment of a patient using a computing device, comprising:
monitoring the patient with a medical monitor using a data acquisition device;
inputting physiological data from the data acquisition device relating to the patient to the computing device; and
determining an appropriate treatment based on the physiological data using a mathematical model wherein the mathematical model does not require anatomic features to be incorporated for an appropriate suggestion to be generated.

36. The method of claim 35, wherein the mathematical model comprises a physiological mathematical model.

37. A patient physiologic monitoring assembly comprising:
a sensor generating a real-time physiologic data stream; and
a controller receiving said real-time physiologic data stream, said controller including a logic adapted to
generate a simulated physiologic data stream in response to a simulated treatment procedure simulated on a physiologic mathematical model;
receive said real-time physiologic data stream in response to a physical treatment procedure;
compare said real-time physiologic data stream to said simulated physiologic data stream; and
generate an alarm when said real-time physiologic data stream diverges from said simulated physiologic data stream.

38. A patient physiologic monitoring assembly as described in claim 37, wherein said alarm is generated when said real-time physiologic data stream crosses a hard threshold relative to said simulated physiologic data stream.

39. A patient physiologic monitoring assembly as described in claim 37, wherein said alarm is generated when said real-time physiologic data stream moves in a direction opposite said simulated physiologic data stream.

40. A patient physiologic monitoring assembly as described in claim 37, wherein said logic is further adapted to coordinate said simulated treatment procedure with said physical treatment procedure.

41. A patient physiologic monitoring assembly as described in claim 37, wherein said logic is further adapted to incorporate a plurality of patient attributes into said physiologic mathematical model.

42. A patient physiologic monitoring assembly as described in claim 37, further comprising an event monitor in communication with said controller, said event monitor signaling the initiation of said physical treatment procedure.

43. A method of patient physiologic monitoring comprising:
generating a simulated physiologic data stream in response to a simulated treatment procedure simulated on a physiologic mathematical model;
receiving a real-time physiologic data stream in response to a physical treatment procedure;
comparing said real-time physiologic data stream to said simulated physiologic data stream; and
generating an alarm when said real-time physiologic data stream diverges from said simulated physiologic data stream.

44. A method of patient physiologic monitoring as described in claim 43, further comprising:
developing said physiologic mathematical model; and
generating a simulated treatment procedure on said physiologic mathematical model.

45. A method of patient physiologic monitoring as described in claim 43, further comprising coordinating said simulated treatment procedure with said physical treatment procedure.

46. A method of patient physiologic monitoring as described in claim 43, further comprising generating a prediction of a physiologic response in response to a proposed treatment procedure.
